(19) United States
(12) Patent Application Publication
(10) Pub. No.: US 2024/0336887 A1
(43) Pub. Date: Oct. 10, 2024

(54) ANTIMICROBIAL BIOACTIVE EXTRACT OBTAINED FROM NITROGEN STRESS-GROWN MICROALGAE

(71) Applicant: ISTANBUL TEKNIK UNIVERSITESI, Istanbul (TR)

(72) Inventors: Ece POLAT, Istanbul (TR); Bahar YAVUZTURK GUL, Istanbul (TR); Mahmut ALTINBAS, Istanbul (TR)

(73) Assignee: ISTANBUL TEKNIK UNIVERSITESI, Istanbul (TR)

(21) Appl. No.: 18/578,778

(22) PCT Filed: Jul. 5, 2022
(86) PCT No.: PCT/TR2022/050701

§ 371 Date: Jan. 12, 2024

(30) Foreign Application Priority Data
Jul. 12, 2021 (TR) 2021/011356

(51) Int. Cl. C12N 1/12; C12N 1/00; C12R 1/89

(52) U.S. Cl. CPC C12N 1/12; C12N 1/005; C12R 2001/89

(57) ABSTRACT

A method of obtaining and producing an antimicrobial bioactive extract from nitrogen stress-grown microalgae is provided. An antimicrobial activity study is carried out with the antimicrobial bioactive extract obtained from the microalgae genus Auxenochlorella protothecoides.

### TECHNICAL FIELD

The invention relates to an antimicrobial bioactive extract from nitrogen stress-grown microalgae and the production method thereof. Antimicrobial activity is studied with the microalgae genus Auxenochlorella protothecoides in the said invention.

### PRIOR ART

Today, anthropogenic carbon dioxide emissions, which are an important issue in the world, can be solved by using microalgae and microalgae are an important research area both in terms of environment and energy generation. Microalgae are single-celled, photo-autotrophic organisms that can adapt to environmental conditions with the ability to fast carbon fixation.

Furthermore, the microalgae have potential bioactive compounds for the pharmaceutical industry and are rich in biologically active secondary and primary metabolites. They contain antifungal, antiviral, anti-enzymatic, or antibiotic compounds. They can produce a large number of compounds that may be effective in antimicrobial activity, such as lipids, indoles, terpenes, acetogens, phenols, fatty acids, and volatile halogenated hydrocarbons.

Different growth media and stress conditions used in the microalgae growing directly affect the produced fatty acid chains and the number of carbon, thereby promoting their use as biodiesel or for different usage purposes. In previous studies, many parameters such as trace elements, changing nutrient amounts, and salinity have been investigated and the production of lipid bioproducts belonging to microalgae in high amounts has been investigated. In previous studies, it has been widely researched that particularly reducing nitrogen in the growth medium of the microalgae creates stress and increases lipid production in the microalgae.

Gram-negative bacteria cannot be eliminated as easily as gram-positive bacteria because of their cell wall structure, and it is known in today's antimicrobial substance studies that most extracts or pure substances do not kill both gram-positive bacteria and gram-negative bacteria. This situation causes problems in gram-negative bacteria-induced infections in studies such as antimicrobial therapy.

Therefore, various developments have been made in the art for the extract belonging to the microorganism grown under stress conditions.

The Chinese patent document numbered CN109234354A in the state of the art discloses a method for screening high-efficiency PUFA microalgae and stress culture. In the said patent document, a method is mentioned, comprising the following steps: separation of algae species; Preliminary screening: extraction of oil and analysis of the fatty acid composition of the candidate algal strains and screening of PUFA-rich algae species based on the fat content and the percentage of PUFAs in the total fatty acid content; selection of nitrogen, phosphorus, and iron as nutritional factors for the stress culture of PUFA-rich algae and measurement of the change in algal cell growth medium and nutrient composition.

The International patent document numbered US2012225472A1 in the state of the art discloses the methods for the status and development of lipids in microalgae. In the said patent document, multiple methods are mentioned by way of examples. It is disclosed in the said patent document that after filtering, the algae biomass is washed with N₂ gas to store in the freezer, sonicated in an ice bath, the test tube is centrifugated for 2-3 minutes>1000 rpm (125×g), the organic layer is evaporated under a slight nitrogen flow and placed in a heating block at about 40° C. while evaporating the solvent.

However, no studies have been found on the changes in the antimicrobial activities of the extracts of the biomass of the microalgae obtained as a result of the absence or deficiency of nitrogen in the nutrient medium of the microalgae.

When the stress culture techniques of the microalgae in the art are studied, it is necessary to develop the extract of the microalgae grown under stress conditions.

### OBJECTS OF THE INVENTION

The object of the present invention is to carry out the production of antimicrobial bioactive extracts obtained from nitrogen stress-grown Auxenochlorella protothecoides microalgae.

### DETAILED DESCRIPTION OF THE INVENTION

The antimicrobial bioactive extract obtained from the Auxenochlorella protothecoides microalgae realized to achieve the objects of the present invention is shown in the accompanying figures.

### These Figures are as Follows;

FIG. 1: The graph of the biomass of the microalgae produced with different nitrogen concentrations (0 mM, 0.8 mM, and 7 mM) from which the antimicrobial bioactive extract of the invention is obtained.

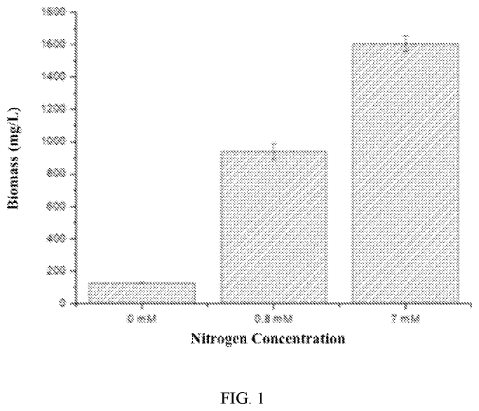

FIG. 2: The graph of the inhibition zone values of the antimicrobial bioactive extract of the invention against gram-positive and gram-negative bacteria based on the nitrogen concentration.

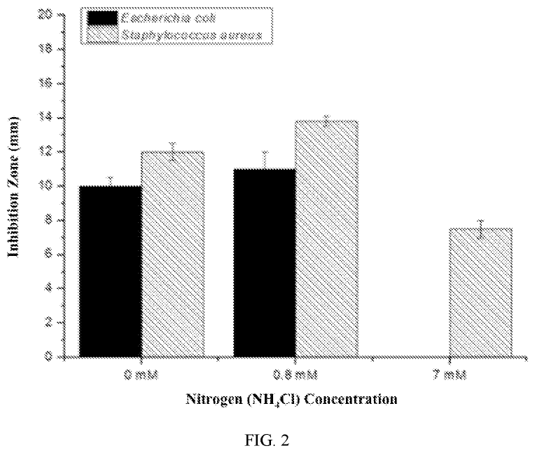

FIG. 3: The graph of the fatty acid methyl ester content of the biomass of the microalgae produced with different nitrogen concentrations (0 mM, 0.8 mM, and 7 mM) from which the antimicrobial bioactive extract of the invention is obtained.

FIG. 4: The graph of the antimicrobial bioactive extract of the invention in the FTIR 2400-4000 cm-1 screening range.

FIG. 5: The graph of the antimicrobial bioactive extract of the invention in the FTIR 650-2000 cm-1 screening range.

FIG. 6: The graph of the antimicrobial bioactive extract of the invention in the FTIR 650-1000 cm-1 screening range.

The invention is an antimicrobial bioactive extract and is characterized in that it contains Auxenochlorella protothecoides microalgae, aliphatic chloro-compounds, phenolic compounds, stearic acid, and palmitic acid, palmitoleic acid, oleic acid, linolenic acid, and linoleic acid.

The invention is an antimicrobial bioactive extract production method, comprising the following steps:
Growing biomass of the Auxenochlorella protothecoides microalgae with nitrogen stress medium,
Lyophilizing the biomass of the microalgae after 5 days of incubation,
Agitating 100 mg lyophilized Auxenochlorella protothecoides microalgae into ethyl acetate overnight,
Then, precipitating with centrifugation at a speed of 10000 rpm,
Separating the supernatant microalgae extract after centrifugation,
Evaporating the solvent with the rotary evaporator at 40±5° C.,
Then, obtaining the extracts by dissolving again with ethyl acetate to obtain concentrations of 10-500 mg/L.

The antimicrobial bioactive extract of the invention contains Auxenochlorella protothecoides microalgae, aliphatic chloro-compounds, phenolic compounds, stearic acid, and palmitic acid, palmitoleic acid, oleic acid, linolenic acid, and linoleic acid.

In previous art studies, bioactive compounds produced by microalgae have not been associated with nitrogen deprivation medium and fatty acid methyl ester composition. In the previous art, it has been reported that the nitrogen stress-dependent biomass production decreased, and in the results obtained in parallel with this it has been observed that the amount of biomass produced with nitrogen stress decreased to 1594 mg/L (stress-free), 942 mg/L (0.8 mM-nitrogen deprivation) and 129 mg/L (0 mM-nitrogen starvation) depending on the increased stress.

The biomass of the microalgae produced with different nitrogen concentrations (0 mM, 0.8 mM, and 7 mM) is used for the extract production of the invention. In the recommended TAP (Tris-Acetate-Phosphate) nutrient medium for the microalgae growing, there is 7 mM NH₄Cl as a nitrogen source. In the extract production of the invention, nitrogen-free stress medium (0 mM) and stress conditions containing approximately 0.11 times more nitrogen (0.8 mM) than TAP (Tris-Acetate-Phosphate) nutrient medium, which is recommended, and one of the standard microalgae growth nutrient media with 7 mM NH₄Cl content, referred to in many microalgae growth studies in the literature, are preferred. The biomass of the microalgae is grown such that a small amount of or no nitrogen is added to the nutrient medium prepared for creating a stress medium. After 5 days of incubation, the biomass of the microalgae is lyophilized.

For the extract production from the biomass of the microalgae according to the invention, 100 mg of lyophilized microalgae is agitated into ethyl acetate overnight. It is then precipitated with centrifugation at a speed of 10000 rpm and the supernatant is separated as the extract of the microalgae. It is dissolved again with ethyl acetate to obtain concentrations of 10-500 mg/L after the solvent is evaporated with a rotary evaporator at 40±5° C.

Auxenochlorella protothecoides microalgae are used as the microalgae genus used in the extract production of the invention.

When the efficiency of the extracts obtained from the biomass of the microalgae using ethyl acetate e solvent against gram-positive (Staphylococcus aureus) and gram-negative (Escherichia coli) bacteria was studied, it was concluded that the extract obtained from the biomass of the microalgae grown in a stress-free medium was not effective in gram-negative bacteria, but the biomass obtained using nitrogen stress exhibited an inhibition effect against both gram-positive and gram-negative bacteria in ethyl acetate extracts. In the results obtained, it was concluded that the ethyl acetate extract obtained from the microalgae genus grown in a medium with a nitrogen content of 0.8 mM was 1.1 times more effective for gram-negative bacteria and 1.15 times more effective for gram-positive bacteria compared to the extract obtained from the biomass of the microalgae grown in nitrogen starvation.

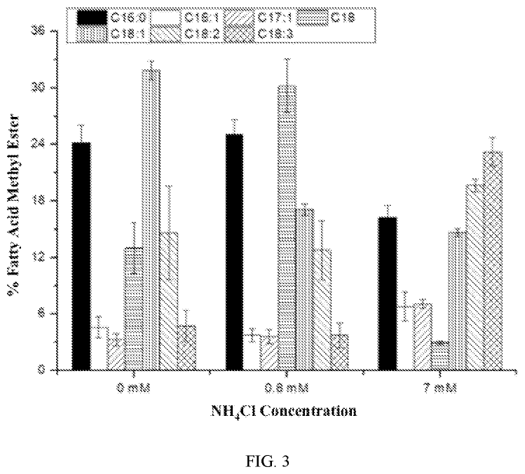

In the analysis of fatty acid methyl ester composition carried out in parallel (FIG. 3), the presence of 2.3 times higher C18: 0 (stearic acid) than nitrogen starvation and 10 times higher than stress-free condition has been detected in the biomass of the microalgae grown in 0.8 mM nitrogen medium. This increase is 1.04 and 1.54 times for C16: 0 (Palmitic acid). Furthermore, palmitoleic acid (C16: 1), oleic acid (C18: 1), linolenic acid (C18: 3) and linoleic acid (C18: 2 9) are also present in the fatty acid methyl ester composition.

Fourier transformation infrared spectrophotometer (FTIR) is a robust method used to detect the types of chemical bonds (functional groups) present in compounds. The wavelength of the absorbed light indicates the characteristic of the chemical bond. The characteristic peaks of the bioactive extracts obtained using the FTIR spectroscope with a scanning range of 650 to 4000 cm⁻¹ were studied in obtaining the extract of the invention.

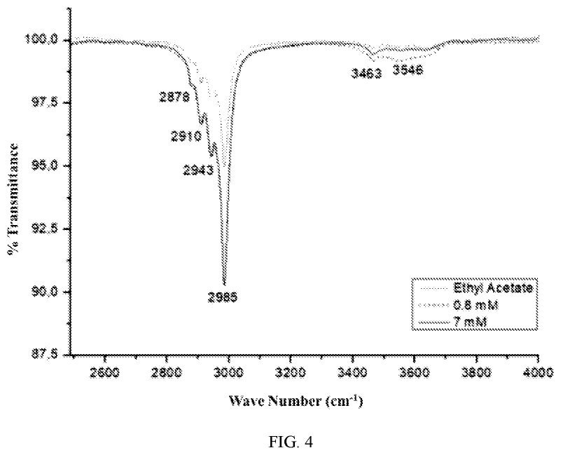

As seen in FIG. 4, the peaks in the 3463 and 3546 cm⁻¹ bands represent O—H stretching vibration and indicate the presence of phenolic compounds. Furthermore, this band exhibits higher transmittance of the extract obtained from the biomass of the microalgae grown under stress (nitrogen deficiency-0.8 mM nitrogen).

The 2985 cm⁻¹ band represents N—H stretching vibration and is a peak that represents amine salts and methyl groups. It indicates unsaturated fatty acid and triacylglycerol.

The 2910 cm⁻¹ and 2878 cm⁻¹ bands represent the CH₂ anti-symmetric stretching of the methyl groups and can be said to represent lipids.

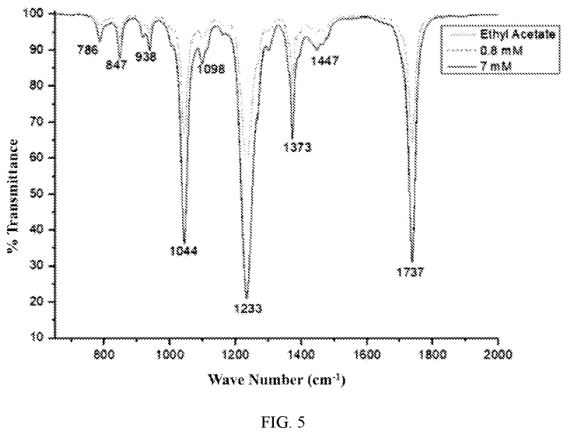

As seen in FIG. 5, there are peaks at 786, 847, 938, 1044, 1098, 1233, 1373, 1447, and 1737 cm⁻¹ wave numbers in the 650-2000 cm⁻¹ scanning range.
The peak at 1737 cm⁻¹ wave number represents lipids.
The peak at 1447 cm⁻¹ wave number represents proteins and lipids.
The peak at 1373 cm⁻¹ wave number represents lipids.
The peak at 1233 cm⁻¹ wave number represents polysaccharides.
Peaks at 1044 and 1098 cm⁻¹ wave numbers represent glycosidic bonds. It may be referred to as the alcoholic group.

These peaks can be associated with bioactive properties compared to ethyl acetate. Antibacterial properties observed in both cases (in extracts obtained from the biomass of the microalgae grown stress-free (7 mM) and under stress (nitrogen deficiency-0.8 mM nitrogen)) against gram-positive bacteria, in particular, can be associated with increased peaks.

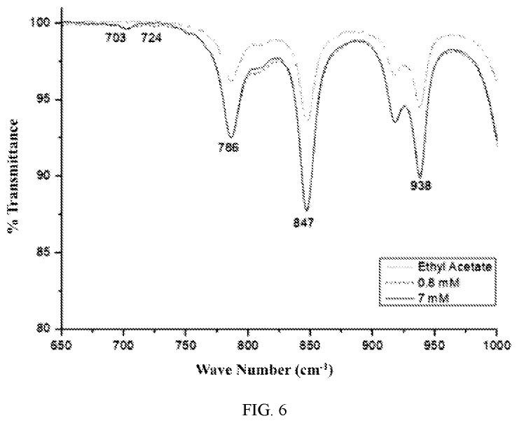

When the peaks observed in the scanning range of 650-1000 cm⁻¹ in FIG. 6 are investigated more closely, they represent the C—Cl stretching vibrations observed at 703 cm⁻¹ and 724 cm⁻¹ wave numbers. This may be explained by the Aliphatic Chloro-compounds present in the extracts' structures. Moreover, it is observed that the one belonging to the 724 cm⁻¹ wave number of these compounds is higher in the extract obtained from the biomass of the microalgae grown under stress (nitrogen deficiency-0.8 mM nitrogen). Similarly, the peaks observed at 786 cm⁻¹ and 847 cm⁻¹ wave numbers represent the aromatic group and are higher than ethyl acetate. Moreover, 938 cm⁻¹ produced a higher peak in aromatic compounds compared to ethyl acetate.

As a result, it is considered that the aliphatic chloro-compounds and phenolic compounds of the extract obtained from the biomass of the microalgae grown under stress (nitrogen deficiency-0.8 mM nitrogen) provide the extract with the ability to be effective against gram-negative bacteria.

The extract of microalgae grown under stress conditions according to the invention can be used as an additive in membrane processes, food, and packaging industries. Besides, the extract of the invention can be used to prevent undesirable pathogenic organisms from damaging the crops during cultivation in agriculture.

What is claimed is:

1. An antimicrobial bioactive extract, wherein the antimicrobial bioactive extract comprises: Auxenochlorella protothecoides microalgae, aliphatic chloro-compounds, phenolic compounds, stearic acid, palmitic acid, palmitoleic acid, oleic acid, linolenic acid, and linoleic acid.

2. A method for producing an antimicrobial bioactive extract, comprising the following steps:
growing a biomass of Auxenochlorella protothecoides microalgae with a medium;
lyophilizing the biomass of Auxenochlorella protothecoides microalgae after 5 days of incubation;
agitating the lyophilized biomass of Auxenochlorella protothecoides microalgae in ethyl acetate overnight;
centrifuging the lyophilized biomass of Auxenochlorella protothecoides microalgae in ethyl acetate at a speed of 10000 rpm to precipitate a pellet;
separating a supernatant microalgae extract after centrifugation;
evaporating the ethyl acetate solvent with a rotary evaporator at 40±5° C.,
dissolving the supernatant microalgae extract again with ethyl acetate to obtain a concentration of 10-500 mg/L.

3. The method for producing an antimicrobial bioactive extract according to claim 2, wherein the medium comprises one selected from the group consisting of a nitrogen-free stress medium, a nitrogen stress medium, and a stress medium containing approximately 0.11 times the nitrogen of the Tris-Acetate-Phosphate (TAP) nutrient medium for the nutrient medium prepared for creating the nitrogen stress medium.
